(19) United States
(12) Patent Application Publication
(10) Pub. No.: US 2003/0087400 A1
(43) Pub. Date: May 8, 2003

(54) Process for the fermentative production of L-lysine using coryneform bacteria

(76) Inventors: Bettina Mockel, Dusseldorf (DE); Walter Pfefferle, Halle (Westf) (DE); Sven Brand, Bielefeld (DE); Alfred Puhler, Bielefeld (DE); Jorn Kalinowski, Bielefeld (DE); Brigitte Bathe, Salzkotten (DE)

Correspondence Address: SMITH, GAMBRELL & RUSSELL, LLP, 1850 M STREET, N.W., SUITE 800, WASHINGTON, DC, 20036, US

(21) Appl. No.: 10/178,219

(22) Filed: Jun. 25, 2002

Related U.S. Application Data
(62) Division of application No. 09/707,913, filed on Nov. 8, 2000.

(30) Foreign Application Priority Data
Nov. 9, 1999 (DE) 199 53 809.3

(51) Int. Cl. C12P 13/08; C12N 9/10; C07H 21/04; C12P 21/02; C12N 1/21; C12N 15/74

(52) U.S. Cl. 435/115; 435/69.1; 435/252.3; 435/193; 435/320.1; 536/23.2

(57) ABSTRACT

The invention relates to a process for the production of L-amino acids, and in particular L-lysine, in which the following steps are performed,

a) fermentation of the bacteria producing the desired L-amino acid, in which at least the csp1 gene is attenuated,

b) accumulation of the desired product in the medium or in the cells of the bacteria and optionally

c) isolation of the L-amino acid,

Bacteria are optionally used in which a further gene of a biosynthetic pathway of the desired L-amino acid is amplified, or in which a metabolic pathway which reduces formation of the desired L-amino acid is at least partially suppressed.

### INTRODUCTION AND BACKGROUND

The present invention provides a process for the fermentative production of L-amino acids, in particular L-lysine, using coryneform bacteria, in which the csp1 gene is attenuated. All references cited herein and throughout this application are expressly incorporated by reference by the term I.B.R. following the citation.

1. Prior art

L-Amino acids, in particular L-lysine, are used in animal nutrition, human medicine and the pharmaceuticals industry. It is known that these amino acids are produced by fermentation of strains of coryneform bacteria, in particular Corynebacterium glutamicum. Due to their great significance, efforts are constantly being made to improve the production process.

Improvements to the process may relate to measures concerning fermentation technology, for example stirring and oxygen supply, or to the composition of the nutrient media, such as for example sugar concentration during fermentation, or to working up of the product by, for example, ion exchange chromatography, or to the intrinsic performance characteristics of the microorganism itself.

The performance characteristics of these microorganisms are improved using methods of mutagenesis, selection and mutant selection. In this manner, strains are obtained which are resistant to antimetabolites, such as for example the lysine analogue S-(2-aminoethyl)cysteine, or are auxotrophic for regulatorily significant metabolites and produce L-amino acids.

For some years, methods of recombinant DNA technology have likewise been used to improve strains of Corynebacterium which produce L-amino acids by amplifying individual biosynthesis genes and investigating the effect on L-amino acid production. Review articles on this subject may be found inter alia in Kinoshita (“Glutamic Acid Bacteria”, in: Biology of Industrial Microorganisms, Demain and Solomon (Eds.), Benjamin Cummings, London, UK, 1985, 115-142) I.B.R., Hilliger (BioTec 2, 40-44 (1991)) I.B.R., Eggeling (Amino Acids 6:261-272 (1994)) I.B.R., Jetten and Sinskey (Critical Reviews in Biotechnology 15, 73-103 (1995)) I.B.R. and Sahm et al. (Annuals of the New York Academy of Science 782, 25-39 (1996)) I.B.R.

L-amino acids, in particular lysine, are used in human medicine and in the pharmaceuticals industry, in the food industry and very particularly in animal nutrition. There is accordingly general interest in providing novel improved processes for the production of amino acids, in particular L-lysine.

Any subsequent mention of L-lysine or lysine should be taken to mean not only the base, but also salts, such as for example lysine monohydrochloride or lysine sulfate.

### SUMMARY OF THE INVENTION

An object of the invention is to provide improved processes for the fermentative production of L-amino acids, and in particular L-lysine, using coryneform bacteria.

### BRIEF DESCRIPTION OF THE FIGURES

FIG. 1 is a Map of the plasmid pKl8mobsacBΔcsp1.

### DETAILED DESCRIPTION OF THE INVENTION

The above and other objects of the invention can be achieved by a process for the fermentative production of L-amino acids, in particular L-lysine, using coryneform bacteria in which at least the nucleotide sequence coding for the Csp1 gene product (csp1 gene) is attenuated. When csp1 gene is attenuated, and in particular expressed at a low level, the desired product is accumulated in the medium or in the cells and the L-amino acid is isolated.

The strains used preferably already produce L-amino acids, and in particular L-lysine, before the csp1 gene is attenuated. In this connection, the term “attenuation” means reducing or suppressing the intracellular activity of one or more enzymes (proteins) in a microorganism, which enzymes are coded by the corresponding DNA (in this case the csp1 gene). For example attenuation may be accomplished by using a weak promoter or a gene or allele which codes for a corresponding enzyme which has a low activity or inactivates the corresponding gene or enzyme (protein) and optionally by combining these measures.

The microorganisms, provided by the present invention, may produce amino acids, in particular lysine, from glucose, sucrose, lactose, fructose, maltose, molasses, starch, cellulose or from glycerol and ethanol. The microorganisms may comprise representatives of the coryneform bacteria in particular of the genus Corynebacterium. Within the genus Corynebacterium, the species Corynebacterium glutamicum may in particular is mentioned, which is known among specialists for its ability to produce L-amino acids.

Suitable strains of the genus Corynebacterium, in particular of the species Corynebacterium glutamicum, are especially the known wild type strains

Corynebacterium glutamicum ATCC13032
Corynebacterium acetoglutainicum ATCC15806
Corynebacterium acetoacidophilum ATCC13870
Corynebacterium melassecola ATCC17965
Corynebacterium thermoaminogenes FERM BP-1539
Brevibacterium flavum ATCC14067
Brevibacterium lactofermentum ATCC13869 and
Brevibacterium divaricatum ATCC14020

also mutants or strains produced therefrom which produce L-amino acids, such as for example the L-lysine producing strains

Corynebacterium glutamicum FERM-P 1709
Brevibacterium flavum EERM-P 1708
Brevibacterium lactofermentum FERM-P 1712
Corynebacterium glutamicum FERM-P 6463
Corynebacterium glutamicum FERN-P 6464 and
Corynebacterium glutamicum DSM 5714.

It has been found that coryneform bacteria produce L-amino acids, in particular L-lysine, in an improved manner once the csp1 gene has been attenuated.

The csp1 gene codes for the PS1 protein, which has not yet been proven to have any enzymatic activity. The nucleotide sequence of the csp1 gene has been described by Joliff et al. (Molecular Microbiology 1992 Aug; 6 (16):2349-62) I.B.R. The sequence is generally available under accession number g40486 from the nucleotide sequence database of the National Center for Biotechnology Information (NCBI, Bethesda, Md., USA) I.B.R. The csp1 gene described in the stated references may be used according to the invention. Alleles of the csp1 gene arising from the degeneracy of the genetic code or from functionally neutral sense mutations may also be used.

Attenuation may be achieved by reducing or suppressing either expression of the csp1 gene or the catalytic properties of the gene product. Both measures are optionally combined.

Gene expression may be reduced by appropriate control of the culture or by genetic modification (mutation) of the signal structures for gene expression. Signal structures for gene expression are, for example, repressor genes, activator genes, operators, promoters, attenuators, ribosome binding sites, the start codon and terminators.

The person skilled in the art will find information in this connection for example in patent application WO 96/15246 I.B.R., in Boyd & Murphy (Journal of Bacteriology 170: 5949 (1988)) I.B.R., in Voskuil & Chambliss (Nucleic Acids Research 26: 3548 (1998)) I.B.R., in Jensen & Hammer (Biotechnology and Bioengineering 58: 191 (1998)) I.B.R., in Patek et al. (Microbiology 142: 1297 (1996)) I.B.R. and in known textbooks of genetics and molecular biology, such as for example the textbook by Knippers (“Molekulare Genetik”, 6th edition, Georg Thieme Verlag, Stuttgart, Germany, 1995) I.B.R. or by Winnacker (“Gene und Klone”, VCH Verlagsgesellschaft, Weinheim, Germany, 1990) I.B.R.

Mutations which give rise to a change or reduction in the catalytic properties of enzyme proteins are known from the prior art; examples which may be mentioned are the papers by Qiu and Goodman (Journal of Biological Chemistry 272: 8611-8617 (1997)) I.B.R., Sugimoto et al. (Bioscience Biotechnology and Biochemistry 61: 1760-1762 (1997)) I.B.R. and Mbckel (“Die Threonindehydratase aus Corynebacterium glutamicum: Aufhebung der allosterischen Regulation und Struktur des Enzyms”, Berichte des Forschungszentrums Julichs, Jul-2906, ISSN09442952, Julich, Germany, 1994) I.B.R.

Summary presentations may be found in known textbooks of genetics and molecular biology such as, for example, the textbook by Hagemann (“Allgemeine Genetik”, Gustav Fischer Verlag, Stuttgart, 1986) I.B.R.

Mutations which-may be considered are transitions, transversions, insertions and deletions. Depending upon the effect of exchanging the amino acids upon enzyme activity, the mutations are known as missense mutations or nonsense mutations. Insertions or deletions of at least one base pair in a gene give rise to frame shift mutations, as a result of which the incorrect amino acids are inserted or translation terminates prematurely. Deletions of two or more codons typically result in a complete breakdown of enzyme activity.

Instructions for producing such mutations belong to the prior art and may be found in known textbooks of genetics and molecular biology, such as for example the textbook by Knippers (“Molekulare Genetik”, 6th edition, Georg Thieme Verlag, Stuttgart, Germany, 1995) I.B.R., by Winnacker (“Gene und Klone”, VCH Verlagsgesellschaft, Weinheim, Germany, 1990) I.B.R. or by Hagemann (“Allgemeine Genetik”; Gustav Fischer Verlag, Stuttgart, 1986) I.B.R.

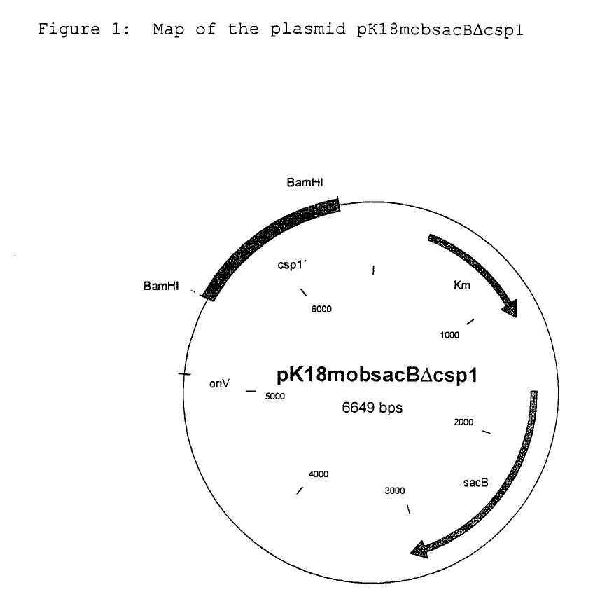

One example of a mutated csp1 gene is the Δcsp1 allele contained in the plasmid pKl8mobsacBΔcsp1 (FIG. 1). The Δcsp1 allele only contains sequences from the 5′ and 3′ ends of the csp1 gene; a section of 1690 bp in length of the coding region is absent (deletion). This Δcsp1 allele may be incorporated into coryneform bacteria by integration mutagenesis.

The above-stated plasmid pKl8mobsacBΔcsp1, which is not replicable in C. glutamicum, is used for this purpose. After transformation and homologous recombination by means of a first “crossing over”, which effects integration, and a second “crossing over”, which effects excision in the csp1 gene, the Δcsp1 deletion is incorporated and a complete loss of function is achieved in the particular strain.

Instructions and explanations relating to integration mutagenesis may be found, for example, in Schwarzer and Pühler (Bio/Technology 9,84-87 (1991)) I.B.R. or Peters-Wendisch et al. (Applied Microbiology 144, 915-927 (1998)) I.B.R.

One example of an amino acid producing strain of coryneform bacteria with an attenuated csp1 gene is the lysine producer Corynebacterium glutamicum R167Δcsp1.

It may additionally be advantageous for the production of L-amino acids, in particular L-lysine, in addition to attenuating the csp1 gene, to amplify one or more enzymes of the particular biosynthetic pathway, of glycolysis, of anaplerotic metabolism, of the citric acid cycle or of amino acid export.

Thus, for example, for the production of L-lysine

the dapA gene (EP-B 0 197 335) I.B.R., which codes for dihydropicolinate synthase, may simultaneously be overexpressed, and/or

the gap gene, which codes for glyceraldehyde 3-phosphate dehydrogenase (Eikmanns (1992), Journal of Bacteriology 174:6076-6086) I.B.R., may simultaneously be overexpressed or

the pyc gene (Eikmanns (1992), Journal of Bacteriology 174:6076-6086) I.B.R., which codes for pyruvate carboxylase, may simultaneously be overexpressed, or

the mqo gene (Molenaar et al., European Journal of Biochemistry 254, 395 - 403 (1998)) I.B.R., which codes for malate:quinone oxidoreductase, may simultaneously be overexpressed, or

the lysE gene (DE-A-195 48 222) I.B.R., which codes for lysine export,

may simultaneously be overexpressed.

It may furthermore be advantageous for the production of amino acids, in particular L-lysine, apart from the csp1 gene, simultaneously to attenuate

the pck gene (DE 199 50 409.1, DSM 13047) I.B.R., which codes for phosphoenolpyruvate carboxykinase, and/or

the pgi gene (US 09/396,478, DSM 12969) I.B.R., which codes for glucose 6-phosphate isomerase.

Finally, it may be advantageous for the production of amino acids, in particular L-lysine, in addition to attenuating the csp1 gene, to suppress unwanted secondary reactions (Nakayama: “Breeding of Amino Acid Producing Micro-organisms”, in: Overproduction of Microbial Products, Krumphanzl, Sikyta, Vanek (eds.), Academic Press, London, UK, 1982) I.B.R.

The culture medium to be used must adequately satisfy the requirements of the particular strains. Culture media for various microorganisms are described in “Manual of Methods for General Bacteriology” from the American Society for Bacteriology (Washington D.C., USA, 1981) I.B.R. Carbon sources which may be used are sugars and carbohydrates, such as glucose, sucrose, lactose, fructose, maltose, molasses, starch and cellulose for example, oils and fats, such as soya oil, sunflower oil, peanut oil and coconut oil for example, fatty acids, such as palmitic acid, stearic acid and linoleic acid for example, alcohols, such as glycerol and ethanol for example, and organic acids, such as acetic acid for example.

These substances may be used individually or as a mixture. Nitrogen sources which may be used comprise organic compounds containing nitrogen, such as peptone-s, yeast extract, meat extract, malt extract, corn steep liquor, soya flour and urea or inorganic compounds, such as ammonium sulfate, ammonium chloride, ammonium phosphate, ammonium carbonate and ammonium nitrate.

The nitrogen sources may be used individually or as a mixture. Phosphorus sources which may be used are phosphoric acid, potassium dihydrogen phosphate or dipotassium hydrogen phosphate or the corresponding salts containing sodium.

The culture medium may additionally contain salts of metals, such as magnesium sulfate or iron sulfate for example, which are necessary for growth. Finally, essential growth-promoting substances such as amino acids and vitamins may also be used in addition to the above-stated substances. Suitable precursors may furthermore be added to the culture medium. The stated feed substances may be added to the culture as a single batch or be fed appropriately during culturing.

Basic compounds, such as sodium hydroxide, potassium hydroxide, ammonia or ammonia water, or acidic compounds, such as phosphoric acid or sulfuric acid, are used appropriately to control the pH of the culture. Foaming may be controlled by using antifoaming agents such as fatty acid polyglycol esters for example.

Plasmid stability may be maintained by the addition to the medium of suitable selectively acting substances, for example antibiotics. Oxygen or oxygen-containing gas mixtures, such as air for example, are introduced into the culture in order to maintain aerobic conditions. The temperature of the culture is normally from 20° C. to 45° C. and preferably from 25° C. to 40° C. The culture is continued until a maximum quantity of the desired product has been formed. This aim is normally achieved within 10 to 160 hours.

Methods for determining L-amino acids are known from the prior art. Analysis may proceed by anion exchange chromatography with subsequent ninhydrin derivatisation, as described in Spackman et al. (Analytical Chemistry, 30, (1958), 1190) I.B.R.or by reversed phase HPLC, as described in Lindroth et al. (Analytical Chemistry (1979) 51: 1167-1174) I.B.R.

The following microorganism has been deposited with Deutsche Sammlung fur Mikroorganismen und Zelikulturen (DSMZ, Braunschweig, Germany) in accordance with the Budapest Treaty:

Escherichia coli strain S17-1 /pKl8mobsacBΔcsp1 as DSM 13048

### EXAMPLES

The present invention is illustrated in greater detail by the following practical examples.

### Example 1

Production of a deletion vector for deletion mutagenesis of the csp1 gene

Chromosomal DNA was isolated from strain ATCC 13032 using the method of Eikmanns et al. (Microbiology 140: 1817-1828 (1994)) I.B.R. The nucleotide sequence of the csp1 gene for C. glutamicum is available under the accession number g404⁸⁶ from the nucleotide sequence database of the National Center for Biotechnology Information (NCBI, Bethesda, Md., USA) I.B.R. On the basis of the known sequence the following oligonucleotides were selected for the polymerase chain reaction:

csp1-10:
5′ CAT CTA G(GA TC)C CGA TGA GCG CCT CCA TGT GT 3′
csp1-11:
5′ GAT CTA G(GA TC)C TCG ACC TTG CGG TGC TGC TT 3′
osp1-del:
5′ GGA ATA CGT AGC CAC CTT CGG TCC CGA AAG TTC CCC
CCT T 3′

The stated primers were synthesised by the company MWG Biotech (Ebersberg, Germany) and the PCR reaction performed in accordance with the standard PCR method of Karreman (BioTechniques 24:736-742, 1998) I.B.R. using Pwo polymerase from Boehringer. The primers csp1-10 and csp1-11 each contain an inserted restriction site for the restriction enzyme BamHI, this site being shown in brackets above. A DNA fragment of approx. 0.9 kb in size, which bears a 1690 bp deletion of the csp1 gene, was isolated with the assistance of the polymerase chain reaction.

The amplified DNA fragment was cut with the restriction enzyme BamHI and purified on an agarose gel (0.8%). The plasmid pKl8mobsacB (Jager et al., Journal of Bacteriology, 1:784-791 (1992)) I.B.R. was also cut with the restriction enzyme BamHI. The plasmid pKl8mobsacB and the PCR fragment were ligated. The E. coli strain S17-1 (Simon et al., 1993, Bio/Technology 1:784-791) I.B.R. was then electroporated with the ligation batch (Hanahan, in DNA cloning. A practical approach. Vol.I. IRL-Press, Oxford, Washington D.C., USA, 1985) I.B.R. Plasmid-bearing cells were selected by plating the transformation batch out onto LB agar (Sambrook et al., Molecular cloning: a laboratory manual. 2^{nd} Ed. Cold Spring Harbor Laboratory Press, Cold Spring Harbor, N.Y., 1989) I.B.R. which had been supplemented with 25 mg/l of kanamycin.

Plasmid DNA was isolated from a transformant using the QIAprep Spin Miniprep Kit from Qiagen and verified by restriction with the restriction enzyme BamHI and subsequent agarose gel electrophoresis (0.8%). The plasmid was named pKl8mobsacBΔcsp1. The strain was designated E. coli S17-1 /pKl8mobsacBΔcsp1and is deposited with Deutsche Sammlung für Mikroorganismen und Zellkulturen (DSMZ, Braunschweig, Germany) under number DSM 13048.

### Example 2

Deletion mutagenesis of csp1 gene into C. glutamicum wild type R167

The vector named pKl8mobsacBΔcsp1 in Example 2 was electroporated into Corynebacterium glutamicum R167 (Liebl et al. (1989) I.B.R. FEMS Microbiological Letters 65:299-304) using the electroporation method of Tauch et al. (FEMS Microbiological Letters, 123:343-347 (1994)) I.B.R. Strain R167 is a restriction-deficient C. glutamicum wild type strain. The vector pKl8mobsacBΔcsp1 cannot independently replicate in C. glutamicum and is only retained in the cell if it has been integrated into the chromosome. Clones with pKl8mobsacBΔcsp1 integrated into the chromosome were selected by plating the electroporation batch out onto LB agar (Sambrook et al., Molecular cloning: a laboratory manual. 2^{nd} Ed. Cold Spring Harbor Laboratory Press, Cold Spring Harbor, N.Y., 1989) I.B.R. which had been supplemented with 15 mg/l of kanamycin. Clones which had grown were plated out onto LB agar with 25 mg/l of kanamycin and incubated for 16 hours at 33° C. In order to achieve excision of the plasmid together with the complete chromosomal copy of the csp1 gene, the clones were then cultured on LB agar with 10% sucrose.

Plasmid pKl8mobsacB contains a copy of the sacB gene, which converts sucrose into levansucrase, which is toxic to C. glutamicum. Thus, the only clones to grow on LB agar with sucrose are those in which the integrated pKl8mobsacBΔcsp1 has in turn been excised.

Excision of the plasmid may be accompanied by the excision of either the complete chromosomal copy of the csp1 gene or the incomplete copy with the internal-deletion. In order to prove that the incomplete copy of csp1 remains in the chromosome, the plasmid pKl8mobsacBΔcsp1 fragment was labelled with the Dig hybridisation kit from Boehringer using the method according to “The DIG System Users Guide for Filter Hybridization” from Boehringer Mannheim GmbH (Mannheim, Germany, 1993) I.B.R.

Chromosomal DNA of a potential deletion mutant was isolated using the method according to Eikmanns et al. (Microbiology 140: 1817-1828 (1994)) I.B.R. and cut in each case with the restriction enzyme EcoRI. The resultant fragments were separated by agarose gel electrophoresis and hybridised at 68° C. using the Dig hybridisation kit from Boehringer. Two hybridising fragments of approx. 6500 bp and approx. 4000 bp were obtained from the control strain, while two hybridising fragments of approx. 6500 bp and approx. 3200 bp were obtained from the mutant.

It could thus be shown that strain R167 has lost its complete copy of the csp1 gene and, instead, now only has the incomplete copy with the deletion of approx. 1690 bp. The strain was designated C. glutamicum R167Δcsp1.

### Example 3

Production of Lysine

The C. glutamicum strain DSM167Δcsp1 obtained in Example 2 was cultured in a nutrient medium suitable for the production of lysine and the lysine content of the culture supernatant was determined.

To this end, the strain was initially incubated for 33 hours at 33° C. on an agar plate. Starting from this agar plate culture, a preculture was inoculated (10 ml of medium in a 100 ml Erlenmeyer flask). The complete medium CgIII was used as the medium for this preculture. The preculture was incubated for 48 hours at 33° C. on a shaker at 240 rpm. A main culture was inoculated from this preculture, such that the initial OD (660 nm) of the main culture was 0.1 OD. Medium MM was used for the main culture.

Medium MM

| | |
|---|---|
| CSL (Corn Steep Liquor) | 5 g/l |
| MOPS | 20 g/l |
| Glucose (separately autoclaved) | 50 g/l |
| Salts: | |
| (NH₄)₂SO₄) | 25 g/l |
| KH₂PO₄ | 0.1 g/l |
| MgSO₄ * 7 H₂O | 1.0 g/l |
| CaCl₂ * 2 H₂O | 10 mg/l |
| FeSO₄ * 7 H₂O | 10 mg/l |
| MnSO₄ * H₂O | 5.0 mg/l |
| Biotin (sterile-filtered) | 0.3 mg/l |
| Thiamine * HCl (sterile-filtered) | 0.2 mg/l |
| CaCO₃ | 25 g/l |

CSL, MOPS and the salt solution were adjusted to pH 7 with ammonia water and autoclaved. The sterile substrate and vitamin solutions, together with the dry-autoclaved CaCO₃, were then added.

Culturing was performed in a volume of 10 ml in a 100 ml Erlenmeyer flask with flow spoilers. Culturing was performed at 33° C. and 80% atmospheric humidity.

After 48 hours, the OD was determined at a measurement wavelength of 660 nm using a Biomek 1000 (Beckmann Instruments GmbH, Munich). The quantity of lysine formed was determined using an amino acid analyser from Eppendorf-BioTronik (Hamburg, Germany) by ion exchange chromatography and post-column derivatisation with ninhydrin detection.

Table 1 shows the result of the test.

TABLE 1

| Strain | OD (660) | Lysine HCl g/l |
|---|---|---|
| R167 | 13.8 | 0.00 |
| R167Δcsp1 | 12.6 | 0.99 |

The following Figures are attached:

FIG. 1: Map of the plasmid pKl8mobsacBΔcsp1.

The abbreviations and names are defined as follows. The lengths stated should be considered to be approximate.

| | |
|---|---|
| sacB: | sacB gene |
| oriV: | replication origin V |
| KmR: | Kanamycin resistance |
| BamHI: | Restriction site of the restriction enzyme BamHI |
| csp1′: | incomplete fragment of the csp1 gene with |
| | an internal 1690 bp deletion |

Further variations and modifications of the present invention will be apparent to those skilled in the art from a reading of the foregoing and are encompassed by the claims appended hereto.

German patent application 199 53 809.3 is relied upon and incorporated herein by reference.

We claim:

1. A process for the production of an L-amino acid comprising the following steps:
a) fermenting bacteria producing the L-amino acid, in which at least the poxB gene is attenuated, and
b) accumulating the L-amino acid in a medium or in cells of the bacteria.

2. The process according to claim 1, further comprising
c) isolating the L-amino acid.

3. The process according to claim 1, wherein the L-amino acid is L-Lysine.

4. The process according to claim 2, wherein the L-amino acid is L-Lysine.

5. The process according to claim 1, wherein a further gene along the biosynthetic pathway of the L-amino acid in the bacteria is amplified.

6. The process according to claim 1, wherein a metabolic pathway that reduces the formation of the L-amino acid in the bacteria is at least partially suppressed.

7. The process according to claim 1, wherein expression of a polynucleotide which codes for the csp1 gene is reduced.

8. The process according to claim 1, wherein a catalytic property of the polypeptide, for which the polynucleotide csp1 codes, is reduced.

9. The process according to claim 1, wherein the integration mutagenesis process by means of a vector comprising pKI8mobsacBΔcsp1 achieves attenuation.

10. The process according to claim 1, wherein L-lysine is produced by fermenting a bacteria in which one or more genes selected from the group consisting of:
a) the dapA gene, which codes for dihydropicolinate synthase,
b) a DNA fragment which imparts S-(2-aminoethyl)cysteine resistance,
c) the pyc gene, which codes for pyruvate carboxylase,
d) the dapD gene, which codes for tetradihydropicolinate succinylase,
e) the dapE gene, which codes for succinyldiaminopimelate desuccinylase,
f) the gap gene, which codes for glyceraldehyde 3-phosphate dehydrogenase,
g) the mqo gene, which codes for malate:quinone oxidoreductase, and
h) the lysE gene, which codes for lysine export, is/are simultaneously overexpressed or amplified.

11. The process according to claim 1, wherein L-lysine is produced by fermenting bacteria in which one or more genes selected from the group consisting of:
a) the pck gene, which codes for phosphoenolpyruvate carboxykinase, and
b) the pgi gene, which codes for glucose 6-phosphate isomerase.
is/are simultaneously attenuated.

12. The process according to claim 1 wherein the bacteria is of the genus Corynebacterium glutamicum.
